(19) United States
(12) Patent Application Publication
(10) Pub. No.: US 2016/0346413 A1
(43) Pub. Date: Dec. 1, 2016

(54) STERILIZATION DEVICE AND STERILIZATION METHOD USING SAME

(71) Applicants: SOUZOUSHA CO., LTD., Nakauonuma-gun (JP); EPC TECHNOLOGY CO., LTD., Kawaguchi-shi (JP); DAINICHI CO., LTD., Sakado-shi (JP); VANTEAN CO., LTD., Sakado-shi (JP); YOKOYAMA CO., LTD., Fujimino-shi (JP)

(72) Inventor: Mitsuru UCHIYAMA, Nakauonuma-gun (JP)

(73) Assignees: SOUZOUSHA CO., LTD., Nakauonuma-gun (JP); EPC TECHNOLOGY CO., LTD., Kawaguchi-shi (JP); DAINICHI CO., LTD., Sakado-shi (JP); VANTEAN CO., LTD., Sakado-shi (JP); YOKOYAMA CO., LTD., Fujimino-shi (JP)

(21) Appl. No.: 14/907,413

(22) PCT Filed: Jan. 21, 2015
(86) PCT No.: PCT/JP2015/051507

§ 371 Date: Jan. 25, 2016

(51) Int. Cl. A61L 2/07

(52) U.S. Cl. CPC A61L 2/07; A61L 2202/11

(57) ABSTRACT

A sterilization device (1) includes a reactor (4) in which calcium oxide and water are reacted; a sterilization space (6) that is filled with water vapor generated in the reactor (4); a sterilization part (5) formed in the sterilization space (6) on which a sterilization object (2) to be sterilized is placed; a device main body (3) that encases the reactor (4), the sterilization space (6) and the sterilization part (5) to bring the sterilization space (6) into an airtight state; a supply unit (9) for supplying combustion gas into the sterilization space (6); and a discharge passage (13) through which a gas mixture in the sterilization space (6) is discharged to the outside.

### TECHNICAL FIELD

The present invention relates to a sterilization device that utilizes water vapor resulting from a reaction of calcium oxide and a sterilization method using the sterilization device. The device and method can be also applied to drying and cooking in addition to sterilization.

### BACKGROUND ART

Water vapor and calcium oxide are utilized for sterilization (disinfection) (refer to Patent Document 1, for example). However, the method described in Patent Document 1 requires a dedicated generator for producing superheated steam. In addition, there is also the demand that sterilization is carried out with water vapor in an airtight space as much as possible because there are various types of sterilization objects. In this case, it is necessary to generate water vapor in an airtight space. It is, however, not practical to accommodate a dedicated generator in an airtight space for this purpose.

### RELATED ART DOCUMENT

### Patent Document

Patent Document 1: JP-A-2013-212106

### SUMMARY OF THE INVENTION

### Problem to be Solved by the Invention

The present invention has been made in view of the above prior art, and it is, therefore, an object of the present invention to provide a sterilization device in which water vapor can be generated to sterilize a sterilization object in an airtight space, and a sterilization method using the sterilization device.

### Means for Solving the Problem

For the purpose of accomplishing the above object, the present invention provides a sterilization device including a reactor in which calcium oxide and water are reacted; a sterilization space that is filled with water vapor generated in the reactor, a sterilization part formed in the sterilization space on which a sterilization object to be sterilized is placed; a device main body that encases the reactor, the sterilization space and the sterilization part to bring the sterilization space into an airtight state; a supply unit for supplying combustion gas into the sterilization space; and a discharge passage through which a gas mixture in the sterilization space is discharged to the outside.

Preferably, the supply unit has a fuel tank for containing fuel, a combustion part in which the fuel is burned, and a combustion gas introduction passage for introducing combustion gas generated in the combustion part into the sterilization space, and the combustion gas introduction passage and the discharge passage are communicated with each other via a communication passage.

Preferably, the supply unit has a fuel tank for containing fuel, an air tank for containing compressed air, a fuel introduction passage and a compressed air introduction passage for introducing the fuel and the compressed air, respectively, into the sterilization space, and an ignition part provided in the sterilization space for ignition of the fuel. It should be noted that the ignition part may be provided in the fuel introduction passage immediately upstream of the sterilization space.

The present invention also provides a sterilization method including a preparation step of placing a sterilization object on the sterilization part and bringing the sterilization space into an airtight state; a water vapor generation step of reacting the calcium oxide and water to generate water vapor in the reactor, and a supply step of supplying the combustion gas into the sterilization space using the supply unit.

Preferably, the sterilization method according to the present invention further includes a circulation step of directing the gas mixture flowing through the discharge passage to flow into the communication passage to supply the gas mixture, together with the combustion gas, into the sterilization space via the combustion gas introduction passage.

### Effect of the Invention

According to the present invention, because the reactor is located in the sterilization space in an airtight state, water vapor can be generated even if the sterilization space is airtight to prevent the sterilization object from being exposed to the outside air and sufficient air is therefore not present therein. In addition, the supply unit can raise the temperature of the generated water vapor to improve the sterilization performance of the water vapor. The present invention has a key advantage of being airtight, and enables a sterilization object to be subjected to a sterilization treatment in an airtight space by generating water vapor in the airtight space.

In addition, because the combustion gas introduction passage and the communication passage are provided, the water vapor in the sterilization space can be circulated and heated with the combustion gas. Thus, the water vapor can be maintained at a high temperature to improve the sterilization efficiency.

In addition, because the fuel is burned in the sterilization space, a higher temperature environment can be directly provided to the water vapor to improve the sterilization efficiency. In addition, because the combustion is carried out in the sterilization space, the combustion time can be adjusted to control the temperature in the sterilization space.

### BRIEF DESCRIPTION OF DRAWINGS

FIG. 1 is a schematic view of a sterilization device according to the present invention.

FIG. 2 is a flowchart of the sterilization device according to the present invention.

FIG. 3 is a schematic view of another sterilization device according to the present invention.

### MODE FOR CARRYING OUT THE INVENTION

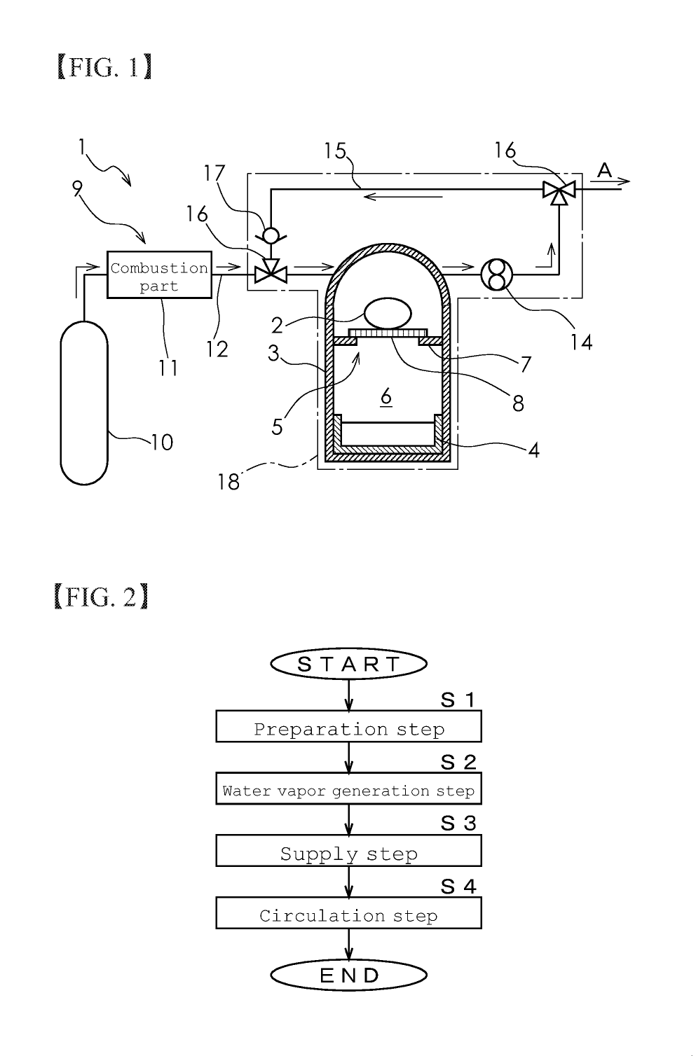

As shown in FIG. 1, a sterilization device 1 according to the present invention includes a device main body 3 that accommodates a sterilization object 2 to be sterilized. A reactor 4 and a sterilization part 5 are arranged in the device main body 3. The device main body 3 defines therein a sterilization space 6 which can be brought into an airtight state. In other words, the device main body 3 encases the reactor 4, the sterilization part 5, and the sterilization space 6. The sterilization object 2 is placed on the sterilization part 5 formed in the sterilization space 6. The sterilization part 5 is formed of support pieces 7 protruding in the device main body 3, and a mesh body 8 placed on the support pieces 7 and allows air to pass through it. The reactor 4 is placed on the bottom of the device main body 3, and calcium oxide and water are supplied into the reactor 4. When the calcium oxide and water react, calcium hydroxide is produced and, at this time, water vapor is generated. The water vapor fills the sterilization space 6. Because the mesh body 8 allows air to pass through it as described above, the water vapor passes through the mesh body 8, directly contacts and sterilizes the sterilization object 2. As the mesh body 8, something that has a generally plate-like or sheet-like shape with a plurality of through holes, such as a metal mesh or punched metal, can be used. The material is not limited to a metal and may be wood or resin.

To prevent pressure rise in the sterilization space 6 due to the generation of water vapor and facilitate combustion of fuel and air, the device main body 3 is provided with a discharge passage 13 in communication with the sterilization space 6. In the example shown in FIG. 1, a structure is shown in which a blower 14 is provided in the discharge passage 13 so that a portion of water vapor can be forcibly discharged to the outside (in the direction of arrow A).

The reaction between calcium oxide and water is as follows:

CaO+H₂O═Ca(OH)₂

The sterilization device 1 also includes a supply unit 9. The supply unit 9 is for supplying combustion gas into the sterilization space 6. In the example shown in FIG. 1, the supply unit has a fuel tank 10, a combustion part 11, and a combustion gas introduction passage 12. The combustion gas introduction passage 12 is a conduit running from the fuel tank 10 through the combustion part 11 to the sterilization space 6 in the device main body 3. Fuel is contained in the fuel tank 10. The fuel is preferably natural gas such as LNG but may be coal or petroleum. The fuel from the fuel tank 10 is supplied into the combustion part 11, and burned therein to generate combustion gas. The combustion gas has a high temperature of several hundred degrees or above, which is higher than the temperature of the water vapor generated from the reactor 4. The combustion gas generated in the combustion part 11 is supplied into the sterilization space 6 through the combustion gas introduction passage. The combustion gas supplied into the sterilization space 6 serves to heat the water vapor in the sterilization space 6 to a higher temperature and maintain it at the temperature.

Here, the combustion gas introduction passage 12 and the discharge passage 13 are communicated with each other via a communication passage 15. Specifically, the communication passage 15 is branched and extended from an intermediate part of the discharge passage 13, and connected to the combustion gas introduction passage 12 downstream of the combustion part 11. A three-way valve 16 is provided at both the branch point and the connection point. Thus, by controlling the three-way valves 16, an airtight circuit 18 consisting of the sterilization space 6, the discharge passage 13, the communication passage 15, and the combustion gas introduction passage 12 can be formed. In particular, an air circulation passage running from the discharge passage through the communication passage 15 to the combustion gas introduction passage 12 can be formed. The water vapor in the sterilization space 6 is directed to flow through the discharge passage 13 by the blower 14, and then flows in the direction of arrow A and is discharged when it is directly discharged to the outside but is directed to flow from an intermediate part of the discharge passage 13 into the combustion gas introduction passage 12 through the communication passage 15 when it is desired to be circulated. When the circulation passage is utilized, the temperature in the sterilization space 6 can be maintained constant by circulating a gas mixture of water vapor and the combustion gas in the sterilization space 6 and heating it with the combustion gas that is supplied as needed. It should be noted that a check valve 17 is provided in the communication passage 15 to prevent the combustion gas from flowing into the discharge passage 13 from the combustion gas introduction passage 12.

When the sterilization object 2 can be sterilized only with the water vapor generated as a result of a reaction between calcium oxide and water, the valve in the combustion gas introduction passage 12 may be closed so that a sterilization treatment can be carried out only with water vapor generated as a result of a chemical reaction between calcium oxide and water.

A sterilization method using the sterilization device 1 as described above is carried out according to the flowchart as shown in FIG. 2. First, a preparation step is carried out (step S1). This preparation step is a step of placing the sterilization object 2 on the sterilization part 5 and bringing the sterilization space 6 into the airtight state. Specifically, a door (not shown) provided on the device main body 3 is opened to place the sterilization object 2 on the sterilization part 5. Then, the door is closed to bring the sterilization space 6 into the airtight state.

Next, a water vapor generation step is carried out (step S2). This water vapor generation step is a step of reacting calcium oxide with water to generate water vapor in the reactor 4. In this step, calcium oxide and water are supplied as needed into the reactor 4. This causes them to react with each other to generate water vapor.

A supply step is also carried out while the water vapor generation step is being carried out (step S3). This supply step is a step of supplying combustion gas into the sterilization space 6 using the supply unit 9. Specifically, the fuel from the fuel tank 10 is burned in the combustion part 11 to generate combustion gas. Then, the combustion gas is supplied through the combustion gas introduction passage 12 into the sterilization space 6.

Because high-temperature water vapor is generated as a result of the above-mentioned water vapor generation step and supply step, the sterilization object 2 is sterilized by the high-temperature water vapor (a gas mixture of the water vapor and combustion gas). To maintain the high temperature of the water vapor, combustion gas is supplied as needed through the combustion gas introduction passage 12.

In addition, a circulation step may be carried out simultaneously with the water vapor generation step and supply step (step S4). This circulation step is a step of directing the gas mixture flowing through the discharge passage 13 to flow into the communication passage 15 and supplying it together with combustion gas into the sterilization space 6 through the combustion gas introduction passage 12. In other words, the gas mixture in the sterilization space 6 is recycled as high-temperature gas in the circulation step. When such a circulation step is used, a sterilization treatment can be carried out in the closed airtight circuit 18. This is advantageous when the sterilization object 2 is not desired to be exposed to the external space.

In the sterilization device 1 and sterilization method described above, because the reactor 4 is located in the sterilization space 6 in the airtight state, water vapor can be generated even if the sterilization space is airtight to prevent the sterilization object 2 from being exposed to the outside air and sufficient air is therefore not present therein. In addition, the supply unit 9 can raise the temperature of the generated water vapor to improve the sterilization performance of the water vapor. The present invention has a key advantage of being airtight, and enables a sterilization object to be subjected to a sterilization treatment in an airtight space (the airtight circuit 18) by generating water vapor in the airtight space.

In addition, because the combustion gas introduction passage 12 and the communication passage 15 are provided, the water vapor in the sterilization space 6 can be circulated and heated with the combustion gas. Thus, the water vapor can be maintained at a high temperature to improve the sterilization efficiency.

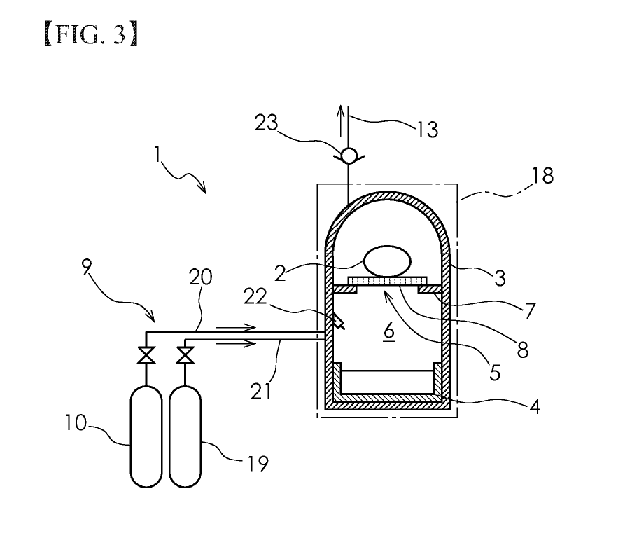

A sterilization device 1 having a structure as shown in FIG. 3 can be also used. In the example shown in FIG. 3, the supply unit 9 and its associated structure are different from those of the device shown in FIG. 1. In other words, the supply unit 9 has a fuel tank 10 and an air tank 19 that are separately disposed and connected to the device main body 3. Specifically, the fuel in the fuel tank 10 is introduced into the sterilization space 6 via a fuel introduction passage 20. The compressed air in the air tank 19 is introduced into the sterilization space 6 via a compressed air introduction passage 21. An ignition part 22 for igniting the fuel is provided in the sterilization space 6. As the ignition part 22, a spark plug, for example, is used. The fuel and compressed air are supplied separately into the sterilization space 6 as described above, and the fuel is burned under the presence of compressed air in the sterilization space 6.

To prevent pressure rise in the sterilization space 6, the device 1 of the example shown in FIG. 3 is also provided with a discharge passage 13 and a one-way valve 23 is provided in an intermediate part of the passage 13. It should be noted that the communication passage 15 as described above may be applied to the device 1 of the example shown in FIG. 3. In this case, the communication passage 15 is connected directly to the device main body 3.

In the device 1 shown in FIG. 3, because the fuel is burned in the sterilization space 6, a higher temperature environment can be directly provided to the water vapor to improve the sterilization efficiency. In addition, because the combustion is carried out in the sterilization space 6, the combustion time can be adjusted to control the temperature in the sterilization space 6. A stirring device for stirring the gas stream may be used in the sterilization device.

### DESCRIPTION OF REFERENCE NUMERALS

1: sterilization device, 2: sterilization object, 3: device main body, 4: reactor, 5: sterilization part, 6: sterilization space, 7: support piece, 8: mesh body, 9: supply unit, 10: fuel tank, 11: combustion part, 12: combustion gas introduction passage, 13: discharge passage, 14: blower, 15: communication passage, 16: three-way valve, 17: check valve, 18: airtight circuit, 19: air tank, 20: fuel introduction passage, 21: compressed air introduction passage, 22: ignition part, 23: one-way valve

1: A sterilization device, comprising:
a reactor in which calcium oxide and water are reacted;
a sterilization space that is filled with water vapor generated in the reactor;
a sterilization part formed in the sterilization space on which a sterilization object to be sterilized is placed;
a device main body that encases the reactor, the sterilization space and the sterilization part to bring the sterilization space into an airtight state;
a supply unit for supplying combustion gas into the sterilization space; and
a discharge passage through which a gas mixture in the sterilization space is discharged to the outside.

2: The sterilization device according to claim 1,
wherein the supply unit has a fuel tank for containing fuel, a combustion part in which the fuel is burned, and a combustion gas introduction passage for introducing combustion gas generated in the combustion part into the sterilization space, and
wherein the combustion gas introduction passage and the discharge passage are communicated with each other via a communication passage.

3: The sterilization device according to claim 1,
wherein the supply unit has a fuel tank for containing fuel, an air tank for containing compressed air, a fuel introduction passage and a compressed air introduction passage for introducing the fuel and the compressed air, respectively, into the sterilization space, and an ignition part provided in the sterilization space for ignition of the fuel.

4: A sterilization method using a sterilization device according to claim 1, comprising:
a preparation step of placing a sterilization object on the sterilization part and bringing the sterilization space into an airtight state;
a water vapor generation step of reacting the calcium oxide and water to generate water vapor in the reactor; and
a supply step of supplying the combustion gas into the sterilization space using the supply unit.

5: The sterilization method according to claim 4,
further comprising a circulation step of directing the gas mixture flowing through the discharge passage to flow into the communication passage to supply the gas mixture, together with the combustion gas, into the sterilization space via the combustion gas introduction passage.

6: A sterilization method using a sterilization device according to claim 2, comprising:
a preparation step of placing a sterilization object on the sterilization part and bringing the sterilization space into an airtight state;
a water vapor generation step of reacting the calcium oxide and water to generate water vapor in the reactor; and
a supply step of supplying the combustion gas into the sterilization space using the supply unit.

7: A sterilization method using a sterilization device according to claim 3, comprising:
a preparation step of placing a sterilization object on the sterilization part and bringing the sterilization space into an airtight state;
a water vapor generation step of reacting the calcium oxide and water to generate water vapor in the reactor; and
a supply step of supplying the combustion gas into the sterilization space using the supply unit.

8: The sterilization method according to claim 6,
further comprising a circulation step of directing the gas mixture flowing through the discharge passage to flow into the communication passage to supply the gas mixture, together with the combustion gas, into the sterilization space via the combustion gas introduction passage.

9: The sterilization method according to claim 7,
further comprising a circulation step of directing the gas mixture flowing through the discharge passage to flow into the communication passage to supply the gas mixture, together with the combustion gas, into the sterilization space via the combustion gas introduction passage.
